(19) United States
(12) Patent Application Publication
(10) Pub. No.: US 2024/0091133 A1
(43) Pub. Date: Mar. 21, 2024

(54) ALCOHOLIC EXTRACT OF CITRUS DEPRESSA PEELS, METHOD FOR OBTAINING IT, AND COSMETIC OR DERMATOLOGICAL COMPOSITION CONTAINING IT

(71) Applicant: CHANEL PARFUMS BEAUTE, Neuilly Sur Seine (FR)

(72) Inventors: Vincent RILHAC, Pantin Cedex (FR); Maeva GILLET, Pantin Cedex (FR)

(73) Assignee: CHANEL PARFUMS BEAUTE, Neuilly Sur Seine (FR)

(21) Appl. No.: 18/467,965

(22) Filed: Sep. 15, 2023

(30) Foreign Application Priority Data
Sep. 16, 2022 (EP) 22196220.2

(51) Int. Cl. A61K 8/9789; A61Q 19/00; A61Q 19/10

(52) U.S. Cl. CPC A61K 8/9789; A61Q 19/002; A61Q 19/10; A61K 2800/80

(57) ABSTRACT

An alcoholic extract of Citrus depressa peels, a cosmetic composition including such an extract having in particular a preventive effect against desquamation of the skin, as well as its method of preparation. Also, the method of limiting/protecting against excessive desquamation and contributing to skin comfort by applying the alcoholic extract of Citrus depressa peels to the skin.

### FIELD

The invention relates to an extract of Citrus depressa peels, a method for obtaining it, a cosmetic or dermatological composition containing it, and its cosmetic use.

### BACKGROUND

The skin is primarily composed of three layers which are, starting from the outermost layer, the epidermis, the dermis, and the hypodermis.

The external layer of the skin, the epidermis, is stratified and greatly contributes to protecting the skin from external aggressions. It is mainly composed of three types of cells, which are keratinocytes (highly predominant), melanocytes, and Langerhans cells. Through its own functions, each of these cell types contributes to the essential role played by the skin in the organism, in particular the role of protecting the organism from external aggressions (climate, ultraviolet rays, tobacco, etc.), called the “barrier function”.

Aging of the epidermis primarily manifests as a reduction in thickness. The atrophy of the epidermis is the consequence of slowed keratinocyte proliferation and the accumulation of senescent keratinocytes. The stratum corneum becomes dull.

Desquamation is a natural phenomenon linked to the fact that the epidermis, which constitutes the upper layer of the skin, is constantly being renewed. The epidermis is composed of several layers of cells, the deepest being the basal layer composed of undifferentiated cells. Over time, these cells will differentiate and migrate to the surface of the epidermis while constituting the various layers therein, until they form corneocytes on the surface of the epidermis, which are dead cells which are eliminated by desquamation. This surface loss is compensated for by the migration of cells from the basal layer to the surface of the epidermis. This is the constant renewal of the skin.

However, too much or irregular desquamation of the stratum corneum cells can lead to formation of large, thick clumps of cells that are visible to the naked eye, and are called “scales” or “dandruff” in the context of the scalp, or in other situations can lead to a thinning of the stratum corneum which cases a feeling of tightness and discomfort. Desquamation disorders, resulting from abnormal or irregular desquamation, can lead to fragility or even a deficiency in the barrier properties of the epidermis.

The presence of scales or dandruff can be frequent, recurrent states, and can be unsightly and clearly uncomfortable.

There is therefore a need to have new active agents which act on excessive desquamation of the skin or scalp in order to limit and/or prevent it, so as to contribute to skin comfort.

Citrus depressa is a citrus fruit from the Rutaceae family, grown in Japan. The generally aqueous extracts of these fruits can be sought for cosmetic use.

For example, document KR 2012/0043288 proposes the use of an extract of Citrus depressa as a whitening agent, stimulating renewal of the skin.

### SUMMARY

The authors of the present invention have now demonstrated, quite surprisingly, that an alcoholic extract of Citrus depressa peels exhibits activity on the mechanisms involved in desquamation, in particular by inhibiting certain enzymes involved in desquamation.

This observation has led to the development of new non-therapeutic cosmetic compositions, particularly useful for all applications where one wishes to improve skin comfort by preventing or limiting desquamation.

According to a first aspect, the invention therefore relates to an alcoholic extract of Citrus depressa peels, comprising a mixture of hesperidin, nobiletin, and isosinensetin.

Another object of the invention is a cosmetic composition comprising, in a physiologically acceptable medium, at least one alcoholic extract of Citrus depressa peels according to the invention.

According to a third aspect, the invention relates to a method of extraction from Citrus depressa peels, comprising the following steps:
a) extraction from Citrus depressa peels that have been previously dried and ground, with at least one alcoholic solvent;
b) filtration of the mixture resulting from step a), and, optionally, decolorization of the obtained filtrate by adsorption on activated charcoal,
c) concentration of the dry extract by evaporation to a dry extract content of between 5 and 20%, preferably between 9 and 12%,
d) the mixture obtained in c) is left to settle for at least 6 hours until 4 distinct phases are obtained;
e) separation of the phases formed in step d) by removal of the two solid and oily lower phases and the essential oil upper phase, and collection of the alcoholic phase comprising the extract of Citrus depressa peels.

Lastly, an object of the invention according to a fourth aspect is the non-therapeutic cosmetic use of an alcoholic extract of Citrus depressa peels as described above, in order to limit/protect against excessive desquamation and contribute to skin comfort.

### BRIEF DESCRIPTION OF THE FIGURES

The FIGURE illustrates the decrease in gene expression of the kallikreins (KLK) KLK5 and KLK7, two serine proteases involved in skin desquamation, after treatment with alcoholic extract of Citrus depressa peels according to the invention at 0.033%.

### DETAILED DESCRIPTION

Citrus depressa

Citrus depressa, also known as shīkwāsā, is a citrus fruit from the Rutaceae family, grown in Japan.

The word shikwasa designates both the plant and the fruit.

It is a small bushy tree about 5 m tall, vigorous and dense and fruit-bearing. White flowering occurs in April (flower diameter 3 cm). The fruit is harvested from October to November.

The fruit is small (diameter 2.5-4 cm, height 2-3 cm, weight 25-60 g), slightly concave at the poles, with a thin pericarp (peel). It contains 8 or 10 especially juicy segments, and seeds. The pulp is soft and gelatinous and the juice is acidic. The essence contained in the pericarp has the scent of a Satsuma Mandarin. The juice is therefore primarily used in the food industry, and the peels for the preparation of essential oils.

The extract according to the invention is obtained from Citrus depressa peels, and preferably obtained after pressing the fruit to obtain the juice.

Alcoholic Extract of Citrus depressa Peels

The alcoholic extract of Citrus depressa peels according to the invention comprises a mixture of hesperidin, nobiletin, and isosinensetin.

The combination of these three molecules of interest, obtained by means of a very specific extraction process, in fact allows reducing the gene expression of the kallikreins (KLK) KLK5 and KLK7, two serine proteases involved in skin desquamation, thus limiting/protecting against excessive desquamation and contributing to skin comfort.

According to a preferred embodiment, the alcoholic extract of Citrus depressa peels according to the invention comprises:
0.1 to 5% by weight of hesperidin, relative to the weight of dry extract, preferably from 0.5 to 2% by weight,
0.1 to 5% by weight of nobiletin, preferably from 1 to 2% by weight,
0.1 to 3% by weight of isosinensetin, preferably from 0.5 to 1% by weight,
the percentages being expressed by weight relative to the weight of dry extract.

Preferably, the hesperidin, nobiletin, and isosinensetin are present in a hesperidin/nobiletin/isosinensetin weight ratio of between 0.5/1/1.5 and 1.5/3/4 and preferably 1.1/1.4/0.7.

Method for Preparing an Alcoholic Extract of Citrus depressa Peels

According to one particular embodiment, an object of the invention is a method of extraction from Citrus depressa peels, comprising the following steps:
a) extraction from Citrus depressa peels that have been previously dried and ground, with at least one alcoholic solvent;
b) filtration of the mixture resulting from step a), and, optionally, decolorization of the obtained filtrate by adsorption on activated charcoal,
c) concentration of the dry extract by evaporation until a dry extract content of between 5 and 20% is reached, preferably between 9 and 12%,
d) the mixture obtained in c) is left to settle for at least 6 hours until 4 distinct phases are obtained;
e) separation of the phases formed in step d) by removal of the two solid and oily lower phases and the essential oil upper phase, and collection of the alcoholic phase comprising the extract of Citrus depressa peels.

Indeed, it is to the credit of the applicant to have demonstrated that by extracting from Citrus depressa peels under very specific conditions, it is possible to obtain an extract enriched in molecules of interest for preventing desquamation, such as hesperidin, nobiletin, and isosinensetin.

The method according to the invention implements a first step a) of extraction from Citrus depressa peels, with at least one alcoholic solvent.

The peels resulting from pressing the fruit are preferably dried and then reduced to a dispersible powder by any grinding process conventionally known to those skilled in the art, for example at room temperature in a knife mill or, according to a preferred embodiment, by low temperature grinding.

Preferably, the dispersible powder of Citrus depressa peels used for preparing the extract according to the invention has an average particle size of less than 500 μm, preferably less than 300 μm.

In step a), the Citrus depressa peels undergo extraction with one or more alcoholic solvents, for example selected among:
C₁-C₄ monoalcohols, for example such as methanol, ethanol, or isopropanol; and
diols, for example such as propylene glycol, 1,3-propanediol, or dipropylene glycol.

Preferably, the alcoholic solvent is a monoalcohol comprising from 2 to 4 carbon atoms, more preferably ethanol.

The extraction is generally carried out by immersing the Citrus depressa peels in one or more of the solvents mentioned above while stirring, at temperatures ranging for example from room temperature to 80° C., for a period of about 30 minutes to 8 hours. Preferably, the extraction of step a) is carried out for a period of between 1 and 5 hours, at a temperature of between 50° C. and 70° C.

In particular, the weight ratio of Citrus depressa peels/alcoholic solvent is between 1/1 and 1/15, and preferably is ⅛.

The extraction step is preferably followed by sieving between 50 μm and 150 μm, preferably at 100 μm.

According to one particular embodiment, extraction step a) is carried out twice. The method of the invention then comprises:
a) a first extraction from Citrus depressa peels that have been previously dried and ground, with an alcoholic solvent, preferably ethanol, at a temperature between 50° C. and 70° C. for 1 hour to 5 hours, then sieving between 50 μm and 150 μm;
a′) a second extraction from the Citrus depressa peel residual obtained in step a), with an alcoholic solvent, preferably ethanol, at a temperature between 50° C. and 70° C. for 1 hour to 5 hours, then sieving between 50 μm and 150 μm.

After step a), the method according to the invention implements filtration step b) preferably carried out to a threshold of 30 μm, preferably 15 μm.

Step b) also optionally comprises decolorization of the obtained filtrate, by adsorption of pigments on activated charcoal. Decolorization of the filtrate on activated charcoal is for example carried out for 1 to 6 hours, preferably for 3 hours, while stirring, at a temperature between 20 and 30° C., preferably at ambient temperature (20° C.)

For decolorization, the activated charcoal content is generally between 20 and 40% by weight relative to the total weight of the dry matter of the extract, preferably around 30% by weight.

At the end of the decolorization, the activated charcoal is removed by filtration to a threshold of 5 μm, preferably 2 μm.

The alcoholic filtrate resulting from step b), possibly decolorized, is then concentrated by evaporating the solvent until a dry extract content is obtained of between 5 and 20%, preferably between 9 and 12% (step c). Evaporation of the solvent can for example be carried out by means of a rotary evaporator or a falling film evaporator.

The concentrated extract from step c) is then left to settle for at least 6 hours until 4 distinct phases are obtained: two solid and oily lower phases, an essential oil upper phase, and an intermediate alcoholic phase comprising the extract of Citrus depressa peels. Settling can for example be carried out in an ampoule.

According to a preferred embodiment, settling step d) is carried out for at least 12 hours, preferably at least 24 hours, and more preferably for 24 to 48 hours.

The settling is carried out at a temperature between 0 and 25° C., preferably between 1 and 10° C., more preferably between 4 and 5° C.

Once settling has completed and the four phases have been obtained, the phase of interest is extracted by removal of the two solid and oily lower phases and the upper phase containing the essential oils, and the alcoholic phase comprising the extract of Citrus depressa peels is collected.

The alcoholic extract of Citrus depressa peels can then be filtered (step f) to a threshold of 5 μm, preferably 2 μm, then diluted in an alcoholic solvent that is different from the extraction solvent used in step a).

The extraction solvent introduced in step a) is then evaporated until a residual concentration of less than 1% of extraction solvent is reached, preferably less than 0.5% (step g).

Finally, the mixture obtained at the end of step g) is left to settle for at least 6 hours, preferably 12 hours, then filtered to a threshold of 5 μm, preferably 2 μm. The settling is carried out, as before, at a temperature between 0 and 25° C., preferably between 1 and 10° C., more preferably between 4 and 5° C.

Thus, according to a preferred embodiment, the method of extraction from Citrus depressa peels comprises the following steps:
a) a first extraction from Citrus depressa peels which have been previously dried and ground, with an alcoholic solvent, preferably ethanol, at a temperature between 50° C. and 70° C. for 1 hour to 5 hours, then sieving between 50 μm and 150 μm;
a′) a second extraction from the Citrus depressa peel residual obtained in step a), with an alcoholic solvent, preferably ethanol, at a temperature between 50° C. and 70° C. for 1 hour to 5 hours, then sieving between 50 μm and 150 μm.
b) filtration of the mixture resulting from step a), and, optionally, decolorization of the obtained filtrate by adsorption on activated charcoal,
c) concentration of the dry extract by evaporation to a dry extract content that is between 5 and 20%, preferably between 9 and 12%,
d) the mixture obtained in c) is left to settle for at least 6 hours until 4 distinct phases are obtained;
e) separation of the phases formed in step d) by removal of the two solid and oily lower phases and the essential oil upper phase, and collection of the alcoholic phase comprising the extract of Citrus depressa peels,
f) filtration of the alcoholic fraction collected in step e) to a threshold of 5 μm, preferably 2 μm, then final dilution of the filtered extract in another alcoholic solvent,
g) evaporation of the extraction solvent introduced in step a) until a residual concentration of less than 1% of extraction solvent is reached, preferably less than 0.5%,
h) the mixture obtained in g) is left to settle for at least 6 hours, then filtered to a threshold of 5 μm, preferably 2 μm.

More preferably still, the method of extraction from Citrus depressa peels comprises the following steps:
a) a first extraction from Citrus depressa peels that have been previously dried and ground, with ethanol, in a weight ratio of Citrus depressa peels/ethanol of ⅛, at a temperature between 50° C. and 70° C. for 1 h to 5 h, then sieving at 100 μm;
a′) a second extraction from the Citrus depressa peel residual obtained in step a), with ethanol, in a weight ratio of Citrus depressa peels/ethanol of ⅛, at a temperature between 50° C. and 70° C. for 1 h to 5 h, then sieving at 100 μm.
b) filtration of the mixture from step a) and decolorization of the obtained filtrate by adsorption on activated charcoal for 3 hours at room temperature,
c) concentration of the dry extract by evaporation to a dry extract content of between 10 and 11%,
d) the mixture obtained in c) is left to settle for at least 12 hours, at a temperature between 4 and 5° C., until 4 distinct phases are obtained;
e) separation of the 4 phases formed in step d) by removal of the two solid and oily lower phases and the essential oil upper phase, and collection of the ethanolic phase comprising the extract of Citrus depressa peels,
f) filtration of the ethanolic fraction collected in step e) to a threshold of 2 μm, then final dilution of the filtered extract in pentylene glycol,
g) evaporation of the extraction ethanol introduced in step a) until a residual ethanol concentration of less than 0.5% is reached,
h) the mixture obtained in g) is left to settle for at least 12 hours at a temperature between 4 and 5° C., then filtered to a threshold of 2 μm.

Cosmetic Composition

A further object of the invention is a cosmetic composition comprising, in a physiologically acceptable medium, at least one alcoholic extract extracted from Citrus depressa peels as described above.

The composition used in accordance with the invention generally comprises, in addition to the extract described above, a physiologically acceptable and preferably cosmetically acceptable medium, meaning it is suitable for use in contact with human skin with no risk of toxicity, incompatibility, instability, allergic response, and in particular which does not cause feelings of discomfort (redness, tightness, tingling).

Advantageously, said cosmetic or dermatological composition may be in the form of a powder, an emulsion, a microemulsion, a nanoemulsion, a suspension, a solution, a lotion, a cream, an aqueous or hydroalcoholic gel, a foam, a serum, an aerosol solution or dispersion, or a dispersion of lipid vesicles.

In the case of an emulsion, it can be a water-in-oil or oil-in-water emulsion.

The cosmetic or dermatological composition according to the invention may also comprise a solvent selected according to the various ingredients and the form of administration.

Examples include water (preferably demineralized water or floral waters), or an alcohol such as ethanol.

Said cosmetic composition may also comprise, in addition to the extract according to the invention, at least one additive that is conventional in the field, for example such as at least one compound selected among an emollient or wetting agent, a gelling and/or thickening agent, a surfactant, an oil, an active agent, a colorant, a preservative, an antioxidant, an active agent, an organic or mineral powder, a sunscreen, and a perfume, and in particular:
one or more wetting agent(s), such as polyols (glycerine, diglycerine, propylene glycol, caprylyl glycol, pentylene glycol, hexanediol), sugars, glycosaminoglycans such as hyaluronic acid and its salts and esters; and polyquaterniums such as lipidure-PMB. Said wetting agent will be present in the composition at a content of about 0 to 30%, preferably 0.005 to 10% by weight of the total weight of the composition.
one or more emollient agent(s), which may be selected for example among esters such as jojoba esters, esters of fatty acids and of fatty alcohol (octyldodecyl myristate, triethylhexanoin, dicaprylyl carbonate, isostearyl isostearate, caprylic/capric triglyceride), butters such as shea butters (butyrospermum parkii butter extract, shea butter ethyl esters, marketed under the names LIPEX SHEASOFT, LIPEX SHEA-U, LIPEX SHEA, LIPEX SHEALIGHT, LIPEX SHEA TRIS) or moringa butters (moringa oil/hydrogenated moringa oil esters), waxes (Acacia decurrens flower wax & Helianthus annuus seed cera seed wax, C10-18 triglycerides), vegetable oils, phytosqualane, alkanes (undecane, tridecane). Said emollient agent will be present in the composition at a content of about 0.1 to 30%, preferably 0.5 to 10%, by weight of the total weight of the composition.
one or more gelling and/or thickening agent(s) for the aqueous phase, selected for example among cellulose derivatives, gums of plant origin (guar, carob, alginates, carrageenans, pectins), of microbial origin (xanthan), clays (laponite), crosslinked or non-crosslinked hydrophilic or amphiphilic homo- and copolymers of acrylamido methylpropane sulfonic acid (AMPS), and/or of acrylamide and/or acrylic acid and/or salts or esters of acrylic acid (marketed under the names ARISTOFLEX AVC, Aristoflex AVS, Aristoflex HMB, SIMULGEL NS, Simulgel EG, Simulgel 600, Simulgel 800, Pemulen, carbopol, Sepiplus 400, Seppimax zen, Sepiplus S, COSMEDIA SP). Said gelling and/or thickening agent will be present in the composition at a content of about 0.1 to 10% by weight of the total weight of the composition.
One or more surfactant(s), in particular:
   anionic surfactants such as isethionates, taurates, sarcosinates, glycinates, glutamates, phosphates (C20-22 alkyl phosphate marketed under the name SENSANOV WR)
   amphoteric surfactants such as betaine derivatives, amphoacetates
   nonionic surfactants such as polyglycerol derivatives, sugar derivatives (derivatives of glucosides or xylosides marketed under the name MONTANOV 68, MONTANOV 202, Montanov 82, MONTANOV L, EASYNOV), lecithins.

Said surfactant will be present at a content of about 0.1 to 15%, preferably 0.5 to 10%, by weight relative to the total weight of the composition;
One or more active agent(s) of natural, biotechnological, or synthetic origin having a biological activity and having efficacy on skin via biological sites, for example selected among vitamins such as vitamin C and its derivatives (ascorbyl glucoside, 3-O-ethyl ascorbic acid, ascorbyl tetraisopalmitate), vitamin A and its derivatives, vitamin E and its derivatives, vitamin B3 or niacinamide, panthenol, trace elements, allantoin, adenosine, peptides (Palmitoyl Tetrapeptide-7, Palmitoyl Tripeptide-1, Palmitoyl Pentapeptide-4, Acetyl Dipeptide-1 Cetyl Ester, Acetyl Tetrapeptide-5 marketed under the name NP RIGIN, MATRIXYL 3000, IDEALIFT, EYESERYL), plant extracts (Glycyrrhiza glabra extract, centella asiatica leaf extract, Secale cereale seed extract), yeast extracts, alpha hydroxy acids such as glycolic or lactic acid, tranexamic acid and its derivatives such as cetyl tranexamic ester, etc. Said active agent will be present in the composition at a content of about 0.1 to 10% by weight of the total weight of the composition.

Other additives usually used in cosmetics may also be present in the composition according to the invention, in particular preservatives, antioxidants, or perfumes well known in the technical field.

The person skilled in the art is able to choose, among all of these possible additives, both the nature and the amount of the ones to be added to the composition, so that said composition retains all of its properties.

The invention also relates to the non-therapeutic cosmetic use of the alcoholic extract of Citrus depressa peels to limit/protect against excessive desquamation and contribute to skin comfort.

In this embodiment, the extract or composition is applied to altered but non-pathological skin.

A further object of the invention is the non-therapeutic cosmetic use of an alcoholic extract of Citrus depressa peels as described above, as an agent for inhibiting gene expression of the kallikreins (KLK) KLK5 and KLK7.

The invention will now be illustrated by the following non-limiting examples.

### Example 1: Inhibiting the Expression of Enzymes Involved in Desquamation in Normal Human Keratinocytes Treated with Alcoholic Extract of Citrus depressa Peels According to the Invention

Extract preparation:
An alcoholic extract of Citrus depressa peels according to the invention was prepared, according to the following steps:
a) 1 kg of dried peels is ground into a powder. Extraction from the peels takes place in 8 kg of 96° ethanol while stirring at 60° C. for 2 hours. The mixture is sieved at 100 μm to collect the peel residual and to set aside the extract.
a′) The peel residual is again subjected to extraction in 8 kg of 96° ethanol while stirring at 60° C. for 2 hours. The mixture is sieved at 100 μm.
b) The liquid fractions from steps a) and a′) are combined then filtered at 15 μm.
The ethanolic fraction is decolorized with 70 g of activated charcoal (30% by mass relative to the dry matter of the extract) while stirring, for 3 hours at room temperature.
The activated charcoal is then removed by filtration at 2 μm.
c) The ethanolic extract is concentrated by evaporating the ethanol in a rotary evaporator or a falling film evaporator to obtain a concentrated extract having a dry matter content of between 10% and 11%.
d) The whole is left to settle at 4° C. in an ampoule for 1 to 2 days.
e) The lower solid and oily phases are removed as well as the upper phase containing essential oils.
f) The ethanolic fraction is collected and filtered at 2 μm. Measurement of the dry matter is carried out on the extract and it contains 135 g dry extract.
1215 g pentylene glycol are added to the ethanolic extract to dilute the final extract to 10% dry matter.
g) The ethanol is completely evaporated in a rotary evaporator or a falling film evaporator (final ethanol concentration of less than 0.5%).
h) The extract is left overnight at 4° C. and filtered at 2 μm. 1350 g of final extract 1701EXT are obtained.

Protocol:

Normal human epidermal keratinocytes from three different donors were inoculated into a 24-well plate and cultured in keratinocyte-SFM (k-SFM) supplemented medium for 48 h at 37° C. and 5% CO₂. The cells were then incubated with or without (untreated condition) 0.033% extract for 48 hours. Each condition was carried out in duplicate. Total RNA extraction was carried out using TriPure Isolation Reagent® according to the protocol recommended by the supplier. The complementary DNAs were synthesized and a transcriptome was produced on the Affymetrix GeneChip Human Transcriptome Array 2.0 chip. Bioinformatic analysis of the genes whose expression is modulated at least by a factor of 2 was carried out with the Ingenuity Pathway Analysis software (IPA®, QIAGEN).

Results:

The extract is able to decrease the transcriptional expression of the kallikreins (KLK) KLK5 and KLK7, two serine proteases involved in skin desquamation. The KLK family comprises 15 isoforms, including KLK5 and KLK7, which are expressed in the upper layers of the skin¹ and are involved in particular in epidermal homeostasis². Indeed, these two enzymes are capable of degrading the superficial corneodesmosomes which ensure cohesion between the corneocytes which make up the stratum corneum.

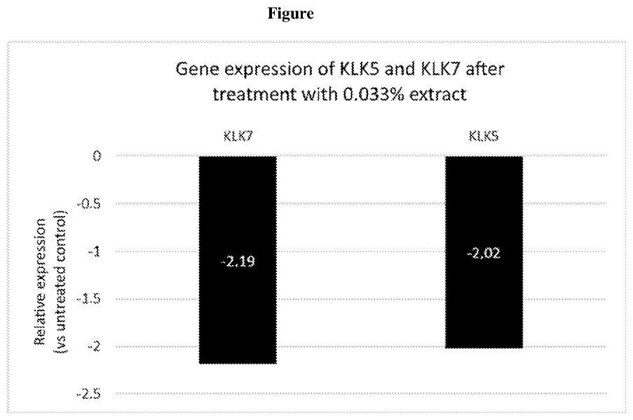

FIG. 1 illustrates the inhibition of the transcriptional expression of KLK5 and KLK7 by the 0.033% extract in normal human keratinocytes.

### Example 2: Cosmetic Compositions

The following compositions can be prepared in a manner that is conventional for those skilled in the art. The quantities indicated below are expressed in percentages by weight. Ingredients in capital letters are identified in accordance with the INCI name.

A—aftershave lotion

| INCI name | (% W/W) |
|---|---|
| Camellia oleifera seed oil | 1-5 |
| Squalane | 1-5 |
| sodium acrylates copolymer & lecithin | 0.1-5 |
| Acrylates/c10-30 alkyl acrylate crosspolymer | 0.1-2 |
| Xanthan gum | 0.01-5 |
| Silica | 0.1-10 |
| Sodium hyaluronate | 0.01-3 |
| Glycerin | 1-30 |
| Alcohol | 1-10 |
| Polyquaternium-51 | 1-10 |
| Allantoin | 0.001-5 |
| Extract of the invention | 0.001-10 |
| yeast extract | 0.1-5 |
| Glycols (Caprylyl Glycol and/or Pentylene Glycol | 0.1-10 |
| and/or Butylene Glycol and/or propanediol) | |
| Water | Qs 100 |

b—aftershave gel

| INCI name | (% w/w) |
|---|---|
| Lauroyl lysine | 1-5 |
| Limnanthes alba (meadowfoam) seed oil | 1-10 |
| Butyrospermum parkii butter (LIPEX SHEASOFT) | 1-10 |
| Butyrospermum parkii butter extract (LIPEX | 1-10 |
| SHEA TRIS) | |
| Camellia oleifera seed oil | 1-10 |
| Extract of the invention | 0.001-10 |
| Squalane | 1-5 |
| Hydrogenated lecithin & glycine soja (soybean) | 1-5 |
| sterols | |
| Ammonium acryloyldimethyltaurate/VP | 1-7 |
| copolymer | |
| Xanthan gum | 0.01-2 |
| Agar | 0.1-5 |
| Yeast extract | 1-3 |
| Saccharide isomerate | 1-5 |
| Adenosine | 0.1-0.5 |
| Niacinamide | 0.1-5 |
| Water | Qs 100 |

c—Shower gel

| INCI name | (% w/w) |
|---|---|
| Sodium methyl cocoyl taurate | 1-15 |
| Sodium methyl oleoyl taurate | 1-10 |
| Sodium cocoyl isethionate | 1-15 |
| Sodium lauroyl sarcosinate | 1-10 |
| Cocamidopropyl betaine | 1-7 |
| Sodium lauroamphoacetate | 1-5 |
| Extract of the invention | 0.001-10 |
| Xanthan Gum | 0.01-2 |
| Yeast extract | 1-3 |
| Saccharide isomerate | 1-5 |
| Water | Qs 100 |

These compositions can be applied every day, morning and/or evening, to the skin. They prevent excessive desquamation and provide user comfort.

1. An alcoholic extract of Citrus depressa peels, comprising a mixture of hesperidin, nobiletin, and isosinensetin.

2. The alcoholic extract according to claim 1, wherein it comprises:
0.1 to 5% by weight of hesperidin, relative to the weight of dry extract, preferably from 0.5 to 2% by weight,
0.1 to 5% by weight of nobiletin, preferably from 1 to 2% by weight,
0.1 to 3% by weight of isosinensetin, preferably from 0.5 to 1% by weight,
the percentages being expressed by weight relative to the weight of dry extract.

3. The alcoholic extract according to claim 1, wherein the hesperidin, nobiletin, and isosinensetin are present in a hesperidin/nobiletin/isosinensetin weight ratio of between 0.5/1/1.5 and 1.5/3/4 and preferably 1.1/1.4/0.7.

4. A cosmetic or dermatological composition comprising, in a cosmetically or pharmaceutically acceptable medium, the alcoholic extract extracted from Citrus depressa peels according to claim 1.

5. The cosmetic or dermatological composition according to claim 4, wherein the cosmetic or dermatological composition is suitable for topical application.

6. A method of extraction from Citrus depressa peels, comprising the following steps:
a) extracting from Citrus depressa peels that have been previously dried and ground, with at least one alcoholic solvent;
b) filtering the mixture resulting from step a), and, optionally, decolorizing the obtained filtrate by adsorption on activated charcoal,
c) concentrating the dry extract by evaporation to a dry extract content of between 5 and 20%, preferably between 9 and 12%,
d) leaving the mixture obtained in c) to settle for at least 6 hours until 4 distinct phases are obtained;
e) separating the phases formed in step d) by removal of the two solid and oily lower phases and the essential oil upper phase, and collection of the alcoholic phase comprising the extract of Citrus depressa peels.

7. The method according to claim 6, wherein step a) is carried out with ethanol, at a temperature between 50° C. and 70° C. for 1 h to 5 h.

8. The method according to claim 6, wherein the extraction is carried out in two stages:
a) a first extraction from Citrus depressa peels that have been previously dried and ground, with ethanol, at a temperature between 50° C. and 70° C. for 1 h to 5 h, then sieving between 50 μm and 150 μm;
a′) a second extraction from the Citrus depressa peel residual obtained in step a), with ethanol, at a temperature between 50° C. and 70° C. for 1 hour to 5 hours, then sieving between 50 μm and 150 μm.

9. The method according to claim 6, wherein filtration step b) is carried out to a threshold of 30 μm, preferably 15 μm, and wherein the decolorization of the filtrate on activated charcoal is carried out for 1 to 6 hours, preferably for 3 hours, then the charcoal is removed by filtration to a threshold of 5 μm, preferably 2 μm.

10. The method according to claim 6, further comprising the following steps:
f) filtering the alcoholic fraction collected in step e), to a threshold of 5 μm, preferably 2 μm, then final diluting of the filtered extract in another alcoholic solvent,
g) evaporating of the extraction solvent introduced in step a) until a residual concentration of less than 1% of extraction solvent is reached, preferably less than 0.5%,
h) leaving the mixture obtained in g) to settle for at least 6 hours, then filtered to a threshold of 5 μm, preferably 2 μm.

11. A method of limiting/protecting against excessive desquamation and contributing to skin comfort of a subject, comprising applying to the skin of a subject in need thereof an alcoholic extract of Citrus depressa peels according to claim 1 to limit/protect against excessive desquamation and contribute to skin comfort of the subject.
